(19) United States
(12) Patent Application Publication
(10) Pub. No.: US 2011/0027234 A1
(43) Pub. Date: Feb. 3, 2011

(54) METHODS FOR IDENTIFICATION AND SELECTION OF HUMAN EMBRYONIC STEM CELL DERIVED CELLS

(75) Inventors: Lior Gepstein, Haifa (IL); Irit Huber, HaCarmel (IL)

Correspondence Address: MARTIN D. MOYNIHAN d/b/a PRTSI, INC., P.O. BOX 16446, ARLINGTON, VA, 22215, US

(73) Assignee: Technion Research & Development Foundation Ltd., Haifa (IL)

(21) Appl. No.: 12/450,764

(22) PCT Filed: Apr. 10, 2008
(86) PCT No.: PCT/IL2008/000500

§ 371 Date: Oct. 20, 2010

Related U.S. Application Data
(60) Provisional application No. 60/907,619, filed on Apr. 11, 2007.

(51) Int. Cl. A61K 35/12; C12N 15/63; C12N 5/10; C12Q 1/68; C12Q 1/02; A61P 9/00

(52) U.S. Cl. 424/93.7; 435/320.1; 435/366; 435/6; 435/29

(57) ABSTRACT

A nucleic acid construct is disclosed, the nucleic acid comprising a polynucleotide comprising a nucleic acid sequence encoding a detectable expression product, the nucleic acid sequence being operably linked to a human tissue specific promoter. A method of lineage tracing of human stem cells and isolated human embryonic stem cell comprising the nucleic acid construct are also disclosed.

### FIELD AND BACKGROUND OF THE INVENTION

The present invention, in some embodiments thereof, relates to expression vectors that may be used for lineage tracing of human embryonic stem cells (hESC). The expression vectors of the present invention may also be used for selecting hESC-derived tissue-specific cells and more particularly, but not exclusively to hESC-derived cardiac cells.

The adult heart has limited regenerative capacity and therefore any significant cardiac cell loss due to ischemia, infection or inflammation may lead to the development of progressive heart failure, one of the leading causes of worldwide morbidity and mortality. Myocardial cell replacement therapy is emerging as a novel therapeutic paradigm for myocardial tissue repair but is hampered by the paucity of cell sources for human cardiomyocytes. Human embryonic stem cells (hESC) may provide a possible solution for this cell sourcing problem. These unique pluripotent stem cells, derived from the inner cell mass of human blastocysts, can be propagated continuously in culture in the undifferentiated state and coaxed to differentiate into a variety of cell lineages (e.g. cardiomyocytes, pancreatic β-cells, neurons).

Cardiomyocyte induction, following hESC differentiation is demonstrated by the appearance of spontaneously contracting areas in three-dimensional differentiating cell aggregates termed embryoid bodies [EBs; Kehat, et al., J Clin Invest (2001) 108, 407-414). Cells isolated from these beating areas display molecular, structural and functional properties of early-stage cardiomyocytes [Kehat, et al., (2001) supra]. Furthermore, the hESC derived cardiomyocytes can form a functional syncytium [Kehat, et al., Circ Res. (2002) 91, 659-661] and can integrate structurally and functionally within preexisting cardiac tissue both in vitro [in co-culturing studies; Kehat et al., Nat Biotechnol (2004) 22, 1282-1289] and in vivo [by serving as a biological pacemaker in animal models of slow heart rate [Xue et al., Circulation (2005) 111, 11-20].

In order to apply hESC in cardiovascular regenerative medicine and in cardiac research, there is a need to identify and derive pure populations of differentiated cardiomyocytes from the heterogeneous cell mixture within the EBs. Derivation of a homogenous cardiac cell population will ultimately depend upon specificity of the cell selection process. Previous methods used for cardiomyocyte selection include physical enrichment by manual dissection of the contracting areas [Kehat, et al., (2002) supra] and partial enrichment of hESC-cardiomyocytes by centrifugation through a Percoll gradient [Xu et al., Circ Res (2002) 91, 501-508].

Recently, several studies have described the use of tissue specific promoters for selection of specific cell lineages. In a murine ESC model, pancreatic beta cells [Soria et al., Diabetes (2000) 49, 157-162], neurons [Andressen et al., Stem Cells (2001) 19, 419-424; Lang et al., Eur J Neurosci (2004) 20, 3209-3221] and cardiomyocytes [Klug et al., J Clin Invest (1996) 98, 216-224; Kolossov et al., J Cell Biol (1998) 143, 2045-2056] were identification and selected. This strategy has also been fine-tuned and utilized in the mouse ESC model to identify early cardiomyocyte precursor cells [Behfar et al., Faseb J (2002) 16, 1558-1566; Hidaka et al., Faseb J (2003) 17, 740-742] and even subpopulations of cardiomyocytes such as ventricular [Muller et al., Faseb J (2000) 14, 2540-2548], atrial [Kolossov et al., Faseb J (2005) 19, 577-579] or pacemaker cells [Kolossov et al., supra; Gassanov et al., Faseb J (2004) 18, 1710-1712].

U.S. Pat. No. 5,928,943 discloses embryonal cardiac muscle cells, their preparation and their use. Specifically, U.S. Pat. No. 5,928,943 teaches a vector system for the modification of the stem cells and for developing a selection method for the transfected cells. This vector system (an adenovirus or an adenovirus-associated virus shuttle vector) comprises two gene constructs: a) a myosin light-chain-2 (MLC-2v) promoter, the reporter gene β-galactosidase and the selectable marker neomycin; and b) a regulatory DNA sequence of the herpes simplex virus thymidine kinase promoter and the selectable marker gene hygromycin. The disclosed cells are contemplated for cell-mediated gene transplant (e.g. for constructing healthy tissue), for investigating substances and for the transfer of therapeutic genes into the myocardium.

U.S. Publication No. 20050208466 discloses a method for selectively isolating or visualizing a target cell differentiated from an embryonic stem (ES) cell. Specifically, U.S. Publication No. 20050208466 teaches infection with an adenovirus comprising two DNA sequences into a non-human embryonic stem cell. The first recombinant DNA comprises a first promoter, a gene having recombinase-recognition sequences on both ends, and a selective marker gene (the first promoter enables the expression of the selective marker in a target cell differentiated from an embryonic stem cell). The second recombinant DNA comprises a second promoter, being a tissue specific promoter (e.g. Nkx2.5, MEF-2, GATA-4, MLC2v), and a recombinase-expressing gene. When an ES cell (which was transfected with both recombinant DNAs) is induced to differentiate, the second promoter is expressed and the recombinase (i.e. Cre) acts to excise a part held by loxP sequences (of the first recombinant DNA). Consequently, the marker gene (e.g. eGFP) is strongly expressed by the first promoter and a specific target cell (e.g. cardiac muscular cell) can be visualized and selected.

### SUMMARY OF THE INVENTION

According to an aspect of some embodiments of the present invention there is provided a nucleic acid construct comprising a polynucleotide comprising a nucleic acid sequence encoding a detectable expression product, the nucleic acid sequence being operably linked to a human tissue specific promoter.

According to an aspect of some embodiments of the present invention there is provided an isolated human embryonic stem cell comprising the nucleic acid construct.

According to an aspect of some embodiments of the present invention there is provided a purified cell population comprising human embryonic stem cells expressing the nucleic acid construct.

According to an aspect of some embodiments of the present invention there is provided a method of lineage tracing of human stem cells. The method comprising introducing the nucleic acid construct into human embryonic stem (ES) cells, culturing the human ES cells under conditions which allow differentiation into a tissue lineage, and detecting expression of the detectable expression product, thereby lineage tracing the human stem cells.

According to an aspect of some embodiments of the present invention there is provided an isolated population of cells generated according to the method of lineage tracing.

According to an aspect of some embodiments of the present invention there is provided a method of treating a myocardial disease in a subject in need thereof, the method comprising administering to the subject a therapeutically effective amount of the isolated cell population, thereby treating the myocardial disease in the subject.

According to an aspect of some embodiments of the present invention there is provided a use of the isolated cell population for the manufacture of a medicament identified for treating a myocardial disease.

According to an aspect of some embodiments of the present invention there is provided a method of identifying a cardiac modulatory agent, the method comprising contacting the isolated cell population with an agent, wherein an alteration in a cardiac phenotype of the cell population is indicative of a modulatory effect of the agent, thereby identifying the cardiac modulatory agent.

According to some embodiments of the invention, the nucleic acid construct further comprises an additional polynucleotide comprising a nucleic acid sequence encoding an antibiotic resistance moiety, the nucleic acid sequence being operably linked to a constitutive promoter.

According to some embodiments of the invention, the human tissue specific promoter comprises a cardiac specific promoter.

According to some embodiments of the invention, the cardiac specific promoter comprises a myosin light-chain-2 (MLC-2v) promoter.

According to some embodiments of the invention, the cardiac specific promoter comprises an atrial natriuretic peptide (ANP) promoter.

According to some embodiments of the invention, the nucleic acid construct comprises a lentivirus backbone.

According to some embodiments of the invention, the lentivirus backbone comprises a PTK 113 backbone.

According to some embodiments of the invention, the human embryonic stem cells comprise a cardiac phenotype.

According to some embodiments of the invention, the cardiac phenotype comprises a functional phenotype.

According to some embodiments of the invention, the functional phenotype comprises an expression of a cardiac marker.

According to some embodiments of the invention, the cardiac marker is selected from the group consisting of cardiac troponin I (cTnI), sarcomeric α-actinin, MLC-2v, MLC-2a, α-MI-IC, MEF-2C, and ANF.

According to some embodiments of the invention, the method further comprises isolating cells exhibiting the expression of the detectable expression product.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E are images characterizing the pluripotent properties of the transgenic hESC lines. FIGS. 1A-C depict colonies of undifferentiated hESC, propagated from the MLC-2v-hESC transgenic line which were stained positive for specific undifferentiated hESC markers: Tra-I-60 (FIG. 1A); SSEA-4 (FIG. 1B); and Oct4 (green nuclei stained with anti-Oct4 antibodies, FIG. 1C); FIG. 1D depicts non-specific nuclei staining with ToPro3 (blue). FIGS. 1C and 1D are double-staining of the same colony. FIG. 1E depicts undifferentiated hESC cells, obtained from the single-cell clones of the transgenic MLC-2V-hESC line, which were injected subcutaneously into SCID mice and were shown to form teratomas. Of note, Ca—cartilage, bv—blood vessels, me—mesenchyme like tissue, ep—heterogeneous epithelium with goblet cells, cu ep—cuboidal epithelium, co ep—columnar epithelium.

FIGS. 2A-C are images showing expression of eGFP under the transcriptional control of MLC-2v promoter in differentiating EBs. FIG. 2A depicts superposition of the transmitted light and fluorescent images. Of note, the EB on the left was not beating and showed no fluorescence, in contrast, the EB on the right comprised a relatively large contracting area (indicated by the black arrows) that displayed positive eGFP fluorescence; FIG. 2B depicts immunostaining of the eGFP expressing EB with anti-cTnI antibodies (red); FIG. 2C depicts high-magnification representation of FIG. 2B. Of note, the eGFP-expressing cells (green) are stained positive for cTnI (red).

FIGS. 2D-E are images showing immunostaining of dispersed cells isolated from the beating EBs. Of note, the individual eGFP-expressing cells (green, FIG. 2D) are stained positive with anti-cTnI antibodies (red, FIG. 2E).

FIGS. 2F-H are images showing immunostaining of dispersed cells isolated from the beating areas, generated during the differentiation of the single-cell transgenic clone. FIG. 2F depicts eGFP-expressing cells; FIG. 2G depicts immunostaining for MHC; and FIG. 2H depicts superposition of the two images.

FIGS. 2I-J are images showing immunostaining of a contracting EB during the differentiation of the single-cell transgenic clones. Note the relatively homogenous and intense eGFP signal (FIG. 2I) and the positive immunostaining for cTnI (FIG. 2J).

FIGS. 3A-C are histograms of FACS analysis showing the typical fluorescence profile of dispersed cells derived from non-transfected EBs (FIG. 3A), EBs derived from the transgenic MLC-2V-eGFP line (FIG. 3B), and similar-stage EBs derived from the single cell clones (FIG. 3C). Of note, a greater number of cardiomyocyte express eGFP in the single-cell clones (FIG. 3C).

FIGS. 3D-I are images showing FACS selection and culturing of eGFP-expressing cells derived from the MLC-2v transgenic line. FIGS. 3D-F are phase contrast (FIG. 3D), fluorescent image (FIG. 3E) and superposition of the two images (FIG. 3F) of unfractionated cells, which were dispersed from the differentiating EBs and did not undergo FACS sorting. Note that some, but not all of the cells, express eGFP. FIGS. 3G-I are the same images, phase contrast (FIG. 3G), fluorescent image (FIG. 3H) and superposition of the two images (FIG. 3I), of cells acquired 10 days following FACS selection of the eGFP positive cells. Note that all cells express eGFP.

FIGS. 4A-C are images showing immunostaining of FACS sorted eGFP-expressing cells. FIG. 4A depicts eGFP expression; FIG. 4B depicts immunostaining of the same cells with an anti-MLC-2v antibody; FIG. 4C depicts superposition of the two immunosignals, wherein the nuclei (depicted in blue) are counterstained with ToPro3. Note that all cells exhibited both eGFP expression and immunostaining with anti-MLC-2v antibodies.

FIG. 4D are RT-PCR images showing undifferentiated hESC (undifferentiated), unfractionated cells derived from beating EBs prior to FACS sorting (unfractionated), FACS selected eGFP-expressing cells (GFP-sorted) and non-selected cell population (non-GFP). Expression of GAPDH, of the cardiac specific genes MLC-2v, MLC-2a and α-MHC, of the endodermal gene a-fetoprotein and of the pluripotent marker Oct4 were observed. Note the expression of the pluripotent marker Oct4 in the undifferentiated hESC and its significant down-regulation in all other groups. Also, note the highest expression of the cardiac specific genes (MLC-2v, MLC-2a, and α-MHC) in the eGFP-selected cells and the lack of significant expression of endodermal (α-fetoprotein) and ectodermal (beta-III-tubulin) markers in the e-GFP selected cells.

FIGS. 5A-C are images showing multielecode array (MEA) mapping of the electrical activation in EBs. The eGFP-expressing EB was dissected and plated on top of the MEA plate (FIG. 5A). Local extracellular potentials could be recorded only in the electrodes directly underlying the eGFP expressing cells (FIG. 5B) but not in the electrodes underlying the non-green areas. The local activation times (LATs) were determined in each recording electrode and were used to generate color-coded high-resolution electrical activation maps (FIG. 5C) depicting the spread of electrical activation. Note the lack of electrical activity in the non-eGFP expressing areas and the presence of relatively fast conduction in dense eGFP expressing areas.

FIGS. 6A-E are images and graphs of whole cell patch-clamp recordings showing the presence of cardiac-specific action potentials in dispersed eGFP-expressing cells. Of note, the morphology of the action potential recorded from the eGFP expressing cells had an “embryonic-like” phenotype which was similar to that recorded from cardiomyocytes isolated from wild-type EBs at the same developmental stage (FIG. 6E).

FIGS. 7A-J are images showing myocardial engraftment of the eGFP-expressing cells. FIG. 7A depicts Hematoxilin and Eosin (H&E) staining of the grafted area depicting the transplanted hESC derived cardiomyocytes within the host rat myocardium; FIGS. 7B-C depict identification of the transplanted cells and their cardiac phenotype during short-term engraftment studies (3 days). Shown is a high-magnification image of the area shown in the box of FIG. 7A. FIG. 7B depicts immunostaining for eGFP (green) and FIG. 7C depicts superposition of the immunostaining results for eGFP (green) and cTnI (red). Note the excellent co-localization results with the eGFP-expressing cells displaying a cardiac-specific phenotype (yellow cells); FIG. 7D depicts H&E staining of the grafted area; FIGS. 7E-F depict high-resolution immunostaining images of the grafted area shown in FIG. 7D. FIG. 7E depicts staining with anti-human mitochondrial antibody. FIG. 7F depicts co-staining with anti-eGFP and anti-human mitochondrial antibodies. Note the co-staining of the grafted cells with anti-eGFP and anti-human mitochondrial antibodies; FIGS. 7G-I depict identification of the transplanted cells and their cardiac phenotype during long-term engraftment (after 4 weeks). FIG. 7I shows the superposition of the results of immunostaining with anti-GFP antibodies (green, FIG. 7G) and anti-sarcomeric α actinin antibodies (red, FIG. 7H). Note that the eGFP-expressing grafted cells are also stained positive for sarcomeric α-actinin (and are therefore yellow in the right panel) as well as evidence for structural maturation of the grafted cells. FIG. 7J depicts confocal immunostaining images of the transplanted eGFP-expressing cardiomyocytes (grafted as cell-clusters) within the ventricular myocardium. The image shows the results of double-staining with anti-Cx43 (red) and anti-GFP (green) antibodies. Note the presence of gap junctions (punctuate immunostaining for Cx43, red) at the interphase (arrows) between the transplanted (green cells) and host cardiomyocytes as well as at lower density within the grafted cell clump (arrow heads). Nuclei were counterstained with ToPro3 (blue).

### DESCRIPTION OF SPECIFIC EMBODIMENTS OF THE INVENTION

The present invention, in some embodiments thereof, relates to expression vectors that may be used for lineage tracing of human embryonic stem cells (hESC). The expression vectors of the present invention may also be used for selecting hESC-derived tissue-specific cells and more particularly, but not exclusively to hESC-derived cardiac cells.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The present inventors have devised an effective tool for identification and selection of cells differentiated from human embryonic stem cells (hESC) using expression constructs comprising tissue specific promoters. As noted in the background section, several researchers have described the use of expression constructs comprising tissue specific promoters for selection of specific cell lineages in non-human (e.g. murine) models. However, until presently no one has been able to identify and select cells from differentiated human embryonic stem cells using such expression constructs. As such, there has been a lack of tools for identification and selection of pure cell populations from differentiated human embryonic stem cells.

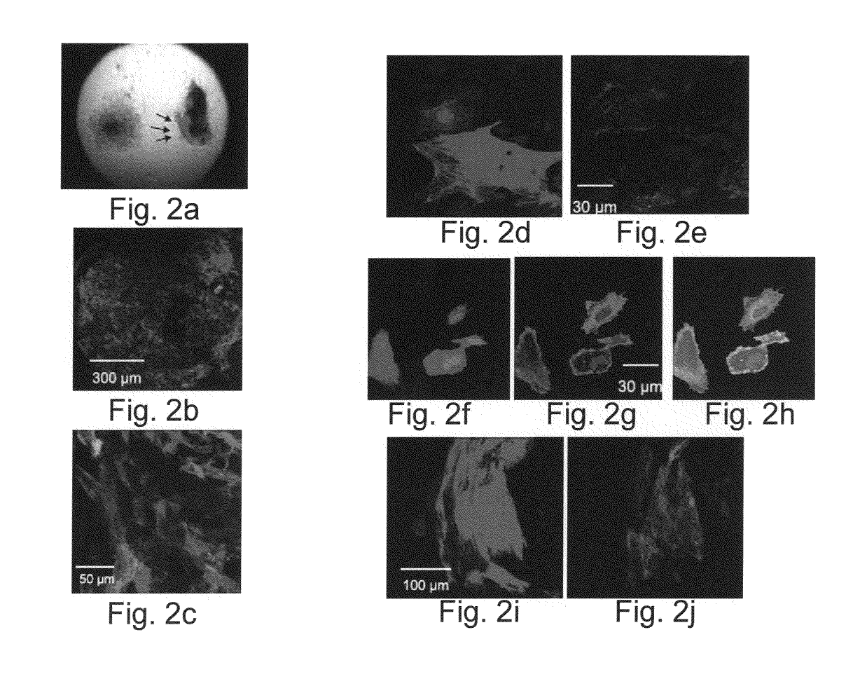

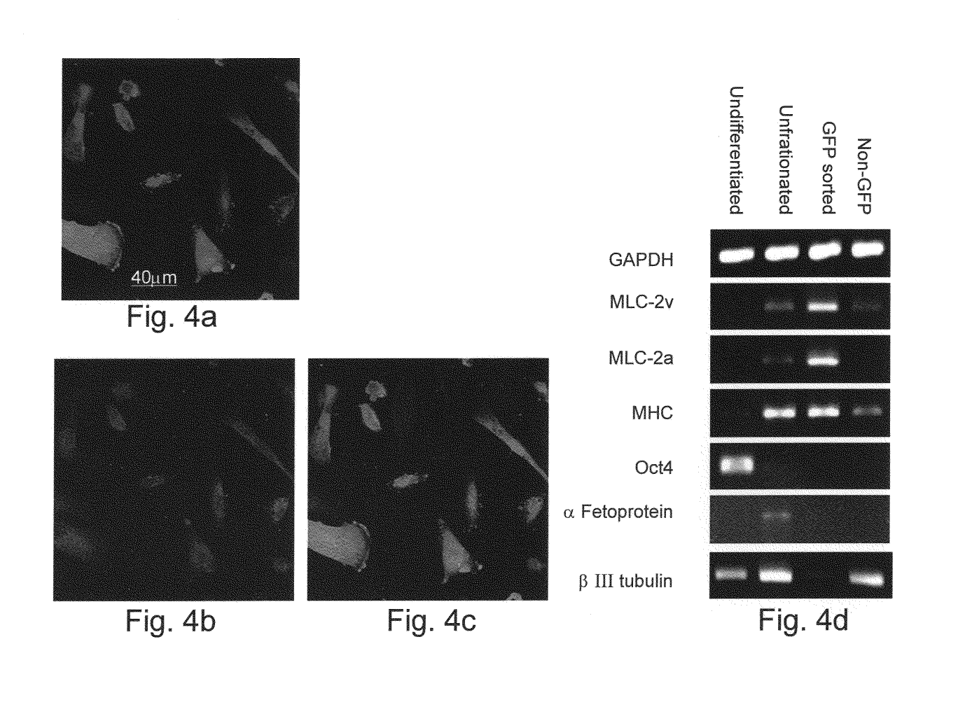

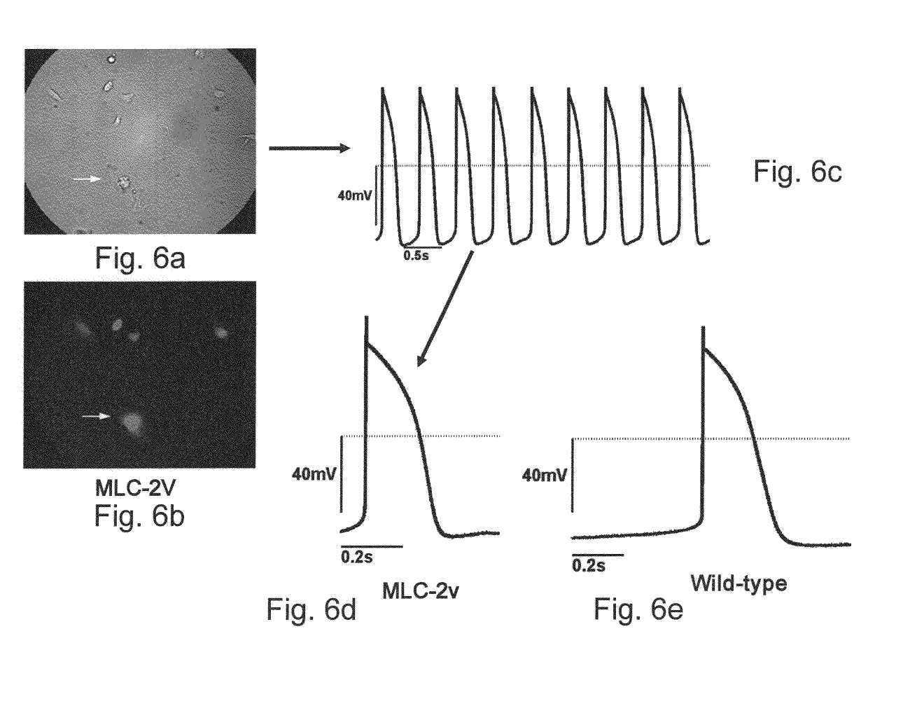

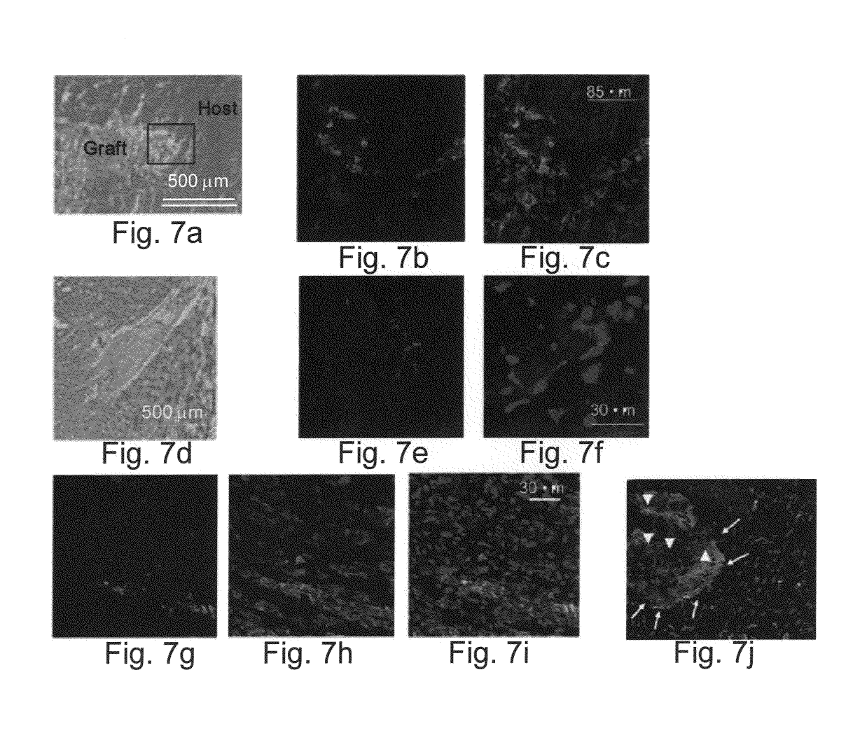

Whilst reducing some embodiments of the present invention to practice, the present inventors have established transgenic hESC lines by introducing lentiviral vectors comprising a cardiac-specific promoter driving the expression of a selectable reporter gene (eGFP). As is illustrated in the Examples section which follows, the present inventors were successful in identifying and selecting hESC-derived cardiomyocytes (depicted as eGFP expressing cells, FIGS. 2A-C and 3G-I) from the in vitro differentiated hESC lines. Moreover, the eGFP expressing cells were shown to express cardiac-specific phenotypes including cardiac-specific proteins (FIGS. 2F-J) and cardiac-specific genes (FIG. 4D) and were further shown to display cardiac-specific action potentials (FIGS. 6A-E). Furthermore, the hESC-derived cardiomyocytes of the present invention formed stable myocardial cell grafts following in vivo cell transplantation (FIGS. 7A-J).

Thus, according to one aspect of the present invention there is provided a method of lineage tracing of human embryonic stem cells. The method comprises introducing a nucleic acid construct comprising a polynucleotide comprising a nucleic acid sequence encoding a detectable expression product, the nucleic acid sequence being operably linked to a human tissue specific promoter, into human embryonic stem (ES) cells. The method further comprises culturing the human ES cells under conditions which allow differentiation into a tissue lineage and detecting expression of the detectable expression product, thereby lineage tracing the human stem cells.

As used herein the phrase “lineage tracing” refers to following and/or identifying the progeny of embryonic stem cells.

Exemplary tissue lineages which may be traced according to the present teachings include, but are not limited to, epithelium tissues (e.g. skin cells, epithelial cells, endothelial cell), connective tissues (e.g. bone cells, blood cells), muscle tissues (e.g. smooth muscle cells, skeletal muscle cells and cardiac muscle cells including, but not limited to, cardiomyocytes, cardiomyocyte precursor cells, ventricular, atrial and pacemaker cells), nervous tissues (e.g. brain cells, spinal cord cells and peripheral nervous system cells), kidney cells, liver cells, lung cells, pancreatic cells, spleen cells, and lymphoid cells (e.g. lymphocytes).

As used herein the phrase “embryonic stem cells” refers to cells from embryonic origin which retain self renewal capability and are capable through their progeny of giving rise to all the cell types which comprise the adult animal including the germ cells. Typically, undifferentiated ES cells have high nuclear/cytoplasmic ratios, prominent nucleoli, and compact colony formation with poorly discernable cell junctions.

Human embryonic stem cells are typically isolated from the blastocyst stage of the human embryos. Human blastocysts are usually obtained from human in vivo preimplantation embryos or from in vitro fertilized (IVF) embryos. Human embryos reach the blastocyst stage 4-5 days post fertilization, at which time they consist of 50-150 cells. Alternatively, a single-cell human embryo can be expanded to the blastocyst stage. For the isolation of human ES cells, the zona pellucida is removed from the blastocyst and the inner cell mass (ICM) is isolated by immunosurgery, in which the trophectoderm cells are lysed and removed from the intact ICM by gentle pipetting. The ICM is then plated in a tissue culture flask containing the appropriate medium enabling its outgrowth. After 9 to 15 days, the ICM-derived outgrowth is dissociated into clumps either mechanically or by an enzymatic degradation, and the cells are then re-plated on a fresh tissue culture medium. Colonies demonstrating undifferentiated morphology are individually selected by micropipette, mechanically dissociated into clumps, and re-plated. Resulting ES cells are then routinely split every 1-2 weeks. For further details on methods of preparation of human ES cells, see: Thomson, J. A. et al. (1998) Science 282, 1145; Thomson, J. A. and Marshall, V. S. (1998) Curr Top Dev Biol 38, 133; Thomson, J. A. et al. (1995). Proc Natl Acad Sci USA 92, 7844, U.S. Pat. No. 5,843,780; Bongso et al. (1989) Hum Reprod 4, 706; and Gardner et al. (1998) Fertil Steril 69, 84.

It will be appreciated that commercially available embryonic stem cells can also be used with this aspect of the present invention. Human ES cells can be purchased from the NIH human embryonic stem cell registry (escr.nih.gov). Non-limiting examples of commercially available embryonic stem cell lines are H9.2, BG01, BG02, BG03, BG04, CY12, CY30, CY92, CY10, TE03, and TE32.

According to this aspect of the present invention, the lineage tracing is effected by introduction of a nucleic acid construct into the human embryonic stem cells.

At its minimum, the nucleic acid construct comprises a human tissue specific promoter which regulates the transcription of a detectable expression product.

As used herein the phrase “detectable expression product” refers to any polypeptide which can be detected in an embryonic stem cell throughout the course of its differentiation without affecting its viability and differentiation capacity.

According to one embodiment, the detectable expression product is a light emitting protein.

Examples of expression products which may be detected in human embryonic stem cells include, but are not limited to, light emitting protein genes such as green fluorescent proteins including EGFP (Enhanced Green Fluorescent Protein) and GFP (Green Fluorescent Protein), blue fluorescent protein (EBFP, EBFP2, Azurite, mKalama1), cyan fluorescent protein (ECFP, Cerulean, CyPet) and yellow fluorescent protein derivatives (YFP, Citrine, Venus, YPet).

The phrase “tissue specific promoter” as used herein refers to a polynucleotide sequence capable of directing expression of a second polynucleotide sequence to which it is operably linked, in a particular tissue or tissues.

Examples of tissue specific promoters include cardiac specific promoters including, but not limited to the promoters of atrial natriuretic peptide (ANP), human myosin light chain-2V (MLC-2v), troponin T (cTnT), Nkx2.5, MEF-2, GATA-4, cardiac muscle-type actin and a-cardiac myosin heavy chain (αMHC) (U.S. Application No. 20050208466); hepatocyte specific promoters including, but not limited to the promoters of albumin of (mature) hepatocytes [Pinkert et al., (1987) Genes Dev. 1:268-277] and α-fetroprotein (AFP) of more undifferentiated hepatocyte (U.S. Application No. 20050208466); lymphoid specific promoters including, but not limited to the promoters of T-cell receptors [Winoto et al., (1989) EMBO J. 8:729-733] and immunoglobulins [Banerji et al. (1983) Cell 33729-740]; neuron specific promoters including, but not limited to the promoters of neurofilament [Byrne et al. (1989) Proc. Natl. Acad. Sci. USA 86:5473-5477], nestin of brain nerve cell and glial fibrillary acidic protein (GFAP) of brain glial cell; other specific promoters include, but not limited to, the promoters of flt-1 of blood vessel (endothelial cell), keratin 14 (K14) of an epidermal keratin cell, and muscle creatine kinase of skeletal muscle cell (U.S. Application No. 20050208466), osteocalcin of osteoblast, pancreas-specific promoters including pancreatic and duodenal homeobox gene 1 (PDX-1) of pancreatic β cell (U.S. Application No. 20050208466) and mammary gland-specific promoters such as the milk whey promoter (U.S. Pat. No. 4,873,316 and European Application Publication No. 264,166).

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

The expression vector of the present invention may also include additional sequences which render it suitable for replication and integration in eukaryotes (e.g., shuttle vectors). Typical cloning vectors contain transcription and translation initiation sequences (e.g., promoters, enhances) and transcription and translation terminators (e.g., polyadenylation signals).

The expression vector of the present invention may further comprise polynucleotide sequences that allow, for example, the translation of several proteins from a single mRNA, such as an internal ribosome entry site (IRES) and sequences for genomic integration of the promoter-chimeric polypeptide.

Thus, according to one embodiment of this aspect of the present invention, the nucleic acid construct of the present invention may also comprise an antibiotic resistance moiety being regulated by a constitutive promoter.

As used herein the term “antibiotic resistance moiety” refers to a polynucleotide encoding a polypeptide that provides antibiotic resistance. According to this embodiment, cells which have been successfully transfected with the expression construct of the present invention are confirmed with resistance to an antibiotic that would normally kill the cell or prevent cell growth. By growing the cells in a medium comprising the antibiotic, it is possible to select the cells which comprise the expression construct of the present invention.

Antibiotic resistance polypeptides include, but are not limited to, β-lactamase, aminoglycoside phosphotransferases, such as neomycin phosphotransferase, chloramphenicol acetyltransferase, the tetracycline resistance protein, the puromycin-resistance protein, hygromycin phosphotransferase, the neomycin resistance protein, the G418 resistance protein and the kanamycin resistance protein.

Constitutive promoters suitable for regulating the antibiotic resistance moieties are promoter sequences that are active at all stages of embryonic stem cell development i.e. both in undifferentiated pluripotent embryonic stem cells and differentiated embryonic stem cells. Examples of constitutive promoters include, but are not limited to the human phosphoglycerate (PGK) promoter, the cytomegalovirus (CMV) promoter, the Rous sarcoma virus (RSV) promoter, the herpes TK promoter, the SV40 early promoter, the SV40 later promoter, the metallothionein promoter, the murine mammary tumor virus promoter, the polyhedrin promoter, or other promoters shown effective for expression in eukaryotic cells.

The nucleic acid construct of the present invention can further include an enhancer, which can be adjacent or distant to the promoter sequence and can function in up regulating the transcription therefrom.

Enhancer elements can stimulate transcription up to 1,000-fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus or human or murine cytomegalovirus (CMV) and the long tandem repeats (LTRs) from various retroviruses, such as murine leukemia virus, murine or Rous sarcoma virus, and HIV. See Gluzman, Y. and Shenk, T., eds. (1983). Enhancers and Eukaryotic Gene Expression, Cold Spring Harbor Press, Cold Spring Harbor, N.Y., which is incorporated herein by reference.

Polyadenylation sequences can also be added to the expression vector of the present invention in order to increase the efficiency of the detectable expression product. Two distinct sequence elements are required for accurate and efficient polyadenylation: GU- or U-rich sequences located downstream from the polyadenylation site and a highly conserved sequence of six nucleotides, namely AAUAAA, located 11-30 nucleotides upstream of the site. Termination and polyadenylation signals suitable for the present invention include those derived from SV40.

In addition to the embodiments already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote extra-chromosomal replication of the viral genome in permissive cell types. Plasmids bearing these viral replicons are replicated episomally as long as the appropriate factors are provided by genes either carried on the plasmid or with the genome of the host cell.

The expression vector of the present invention may or may not include a eukaryotic replicon. If a eukaryotic replicon is present, the vector is capable of amplification in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Examples of mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, and pNMT81, which are available from Invitrogen, pCI which is available from Promega, pMbac, pPbac, pBK-RSV and pBK-CMV, which are available from Strategene, pTRES which is available from Clontech, and their derivatives.

Expression vectors containing regulatory elements from eukaryotic viruses such as retroviruses can be also used. SV40 vectors include pSVT7 and pMT2, for instance. Vectors derived from bovine papilloma virus include pBV-1MTHA, and vectors derived from Epstein-Barr virus include pHEBO and p2O5. Other exemplary vectors include pMSG, pAV009/A⁺, pMTO10/A⁺, pMAMneo-5 and baculovirus pDSVE.

Retroviral vectors represent a class of vectors particularly suitable for use with the present invention. Defective retroviruses are routinely used in transfer of genes into mammalian cells (for a review, see Miller, A. D. (1990). Blood 76, 271). A recombinant retrovirus including a polynucleotide encoding a detectable expression product and/or antibiotic resistance moiety of the present invention can be constructed using well-known molecular techniques. Portions of the retroviral genome can be removed to render the retrovirus replication machinery defective, and the replication-deficient retrovirus can then packaged into virions, which can be used to infect target cells through the use of a helper virus while employing standard techniques. Protocols for producing recombinant retroviruses and for infecting cells with viruses in vitro or in vivo can be found in, for example, Ausubel et al. (1994) Current Protocols in Molecular Biology (Greene Publishing Associates, Inc. & John Wiley & Sons, Inc.). Retroviruses have been used to introduce a variety of genes into many different cell types, including neuronal cells, epithelial cells, endothelial cells, lymphocytes, myoblasts, hepatocytes, and bone marrow cells.

According to one embodiment, a lentiviral vector, a type of retroviral vector, is used according to the present teachings. Lentiviral vectors are widely used as vectors due to their ability to integrate into the genome of non-dividing as well as dividing cells. The viral genome, in the form of RNA, is reverse-transcribed when the virus enters the cell to produce DNA, which is then inserted into the genome at a random position by the viral integrase enzyme. The vector (a provirus) remains in the genome and is passed on to the progeny of the cell when it divides. For safety reasons, lentiviral vectors never carry the genes required for their replication. To produce a lentivirus, several plasmids are transfected into a so-called packaging cell line, commonly HEK 293. One or more plasmids, generally referred to as packaging plasmids, encode the virion proteins, such as the capsid and the reverse transcriptase. Another plasmid contains the genetic material to be delivered by the vector. It is transcribed to produce the single-stranded RNA viral genome and is marked by the presence of the ψ (psi) sequence. This sequence is used to package the genome into the virion.

A specific example of a suitable lentiviral vector for introducing and expressing the polynucleotide sequences of the present invention in a human embryonic stem cell is the lentivirus PTK 113 vector.

Another suitable expression vector that may be used according to this aspect of the present invention is the adenovirus vector. The adenovirus is an extensively studied and routinely used gene transfer vector. Key advantages of an adenovirus vector include relatively high transduction efficiency of dividing and quiescent cells, natural tropism to a to wide range of epithelial tissues, and easy production of high titers (Russel, W. C. (2000) J Gen Virol 81, 57-63). The adenovirus DNA is transported to the nucleus, but does not integrate thereinto. Thus the risk of mutagenesis with adenoviral vectors is minimized, while short-term expression is particularly suitable for treating cancer cells. Adenoviral vectors used in experimental cancer treatments are described by Seth et al. (1999). “Adenoviral vectors for cancer gene therapy,” pp. 103-120, P. Seth, ed., Adenoviruses: Basic Biology to Gene Therapy, Landes, Austin, Tex.).

A suitable viral expression vector may also be a chimeric adenovirus/retrovirus vector combining retroviral and adenoviral components. Such vectors may be more efficient than traditional expression vectors for transducing tumor cells (Pan et al. (2002). Cancer Letts 184, 179-188).

Various methods can be used to introduce the expression vectors of the present invention into human embryonic stem cells. Such methods are generally described in, for instance: Sambrook, J. and Russell, D. W. (1989, 1992, 2001), Molecular Cloning: A Laboratory Manual, Cold Springs Harbor Laboratory, New York; Ausubel, R. M. et al., eds. (1994, 1989). Current Protocols in Molecular Biology, John Wiley and Sons, Baltimore, Md. (1989); Chang, P. L., ed. (1995). Somatic Gene Therapy, CRC Press, Boca Raton, Fla.; Vega, M. A. (1995). Gene Targeting, CRC Press, Boca Raton, Fla.; Rodriguez, R. L. and Denhardt, D. H. (1987). Vectors: A Survey of Molecular Cloning Vectors and Their Uses, Butterworth-Heinemann, Boston, Mass.; and Gilboa, E. et al. (1986). Transfer and expression of cloned genes using retro-viral vectors. Biotechniques 4(6), 504-512; and include, for example, stable or transient transfection, lipofection, electroporation, and infection with recombinant viral vectors. In addition, see U.S. Pat. Nos. 5,464,764 and 5,487,992 for positive-negative selection methods.

Introduction of the expression vectors of the present invention into embryonic stem cells by viral infection offers several advantages over other methods such as lipofection and electroporation. Thus, viral vectors offer higher efficiency of transformation and targeting to, and propagation in, specific cell types.

It will be appreciated that the expression construct of the present invention may be administered alone or together with other expression constructs such as those comprising antibiotic resistance moieties.

Prior to introduction of the expression vector of the present invention, undifferentiated human ES cells are typically cultured using conditioned medium which comprises factors needed for stem cell proliferation while at the same time inhibit their differentiation. Conditioned media can be collected from a variety of cells forming monolayers (i.e., feeder cells) in culture. Examples include mouse embryonic fibroblast (MEF)-conditioned medium, foreskin-conditioned medium, human embryonic fibroblast-conditioned medium, human fallopian epithelial cell-conditioned medium, and others. The growth medium can be supplemented with nutritional factors, such as amino acids (e.g., L-glutamine), anti-oxidants (e.g., beta-mercaptoethanol), and growth factors, which benefit stem cell growth in an undifferentiated state. Serum and serum replacements are added at effective concentration ranges, as described elsewhere (U.S. patent application Ser. No. 10/368,045).

Currently practiced ES cell culturing methods are mainly based on the use of feeder cell layers which secrete factors needed for stem cell proliferation. Commonly used feeder cell layers include mouse feeder layers, foreskin feeder layers and human embryonic fibroblasts or adult fallopian epithelial cells as feeder cell layers. Feeder cell-free systems can also be used in ES cell culturing, utilizing matrices supplemented with serum, cytokines, and growth factors as a replacement for the feeder cell layer.

As mentioned, following introduction of the expression vector of the present invention into the human embryonic stem cells, the cells are cultured under conditions which allow differentiation into a tissue lineage.

To enable differentiation of human ES cells into specific tissues, the culturing conditions described above are specifically modified. For example, to enable differentiation of ES cells into tissue specific cells, the cells may first be differentiated into embryoid bodies (EBs) by removal of ES cells from feeder layers or feeder cell-free culture systems. ES cell removal can be effected using collagenase treatment (e.g. type IV) which enables dispersion of the hESCs into small clumps of 3-20 cells (see Example 1, hereinbelow). Following dissociation from the culturing surface, the cells may be cultivated in suspension for 7 to 10 days and aggregated to form EBs (see Example 1, hereinbelow). The EBs may then be transferred to tissue culture plates (e.g. gelatin coated culture plates) containing a culture medium supplemented with factors that induce further differentiation. For example, as illustrated in the Example section herein below, serum and amino acids may be added to differentiate the EBs towards a cardiac lineage.

According to another embodiment, the embryonic stem cells are grown under adherent conditions without the formation of embryoid bodies in the presence of growth factors and other differentiation agents in order to enable differentiation of ES cells into tissue specific cells.

According to yet another embodiment, the embryonic stem cells are grown in suspension whilst differentiating the hES cells in the presence of growth factors and other differentiation agents.

Exemplary growth factors and differentiation agents that may be used to differentiate the human embryonic stem cells of the present invention into specialized cells (e.g. insulin secreting cells, brain cells, muscle cells, cardiac cells) include, but are not limited to basic fibroblast growth factor (bFGF), transforming growth factor beta1 (TGF-beta1), activin-A, bone morphogenic protein 4 (BMP-4), hepatocyte growth factor (HGF), epidermal growth factor (EGF), beta nerve growth factor (betaNGF), and retinoic acid. Specifically, Activin-A and TGFbeta1 mainly induce differentiation into mesodermal cells; retinoic acid, EGF, BMP-4, and bFGF activate ectodermal and mesodermal cell differentiation; and NGF and HGF allow differentiation into the three embryonic germ layers [Schuldiner et al., Proc Natl Acad Sci USA. (2000) 97(21):11307-12].

Following culturing, the cells are typically analyzed for expression of the detectable product.

Any method known in the art can be utilized for detecting cells which express the detectable expression product. For example, a method for detecting expression of a LacZ gene (which encodes β-galactosidase (LacZ), an intracellular enzyme that cleaves the disaccharide lactose into glucose and galactose) by x-gal staining of a tissue utilizes an enzymatic reaction, detection sensitivity is relatively high, and a level of expression of a LacZ gene necessary for detection may be very low, however a LacZ gene cannot be used as a marker gene for live cells. For this reason, it is preferable to use a light emitting protein (e.g. EGFP) which enables visualization of EGFP with a fluorescent microscope or enables separation with a cell sorter (i.e. by flow cytometry).

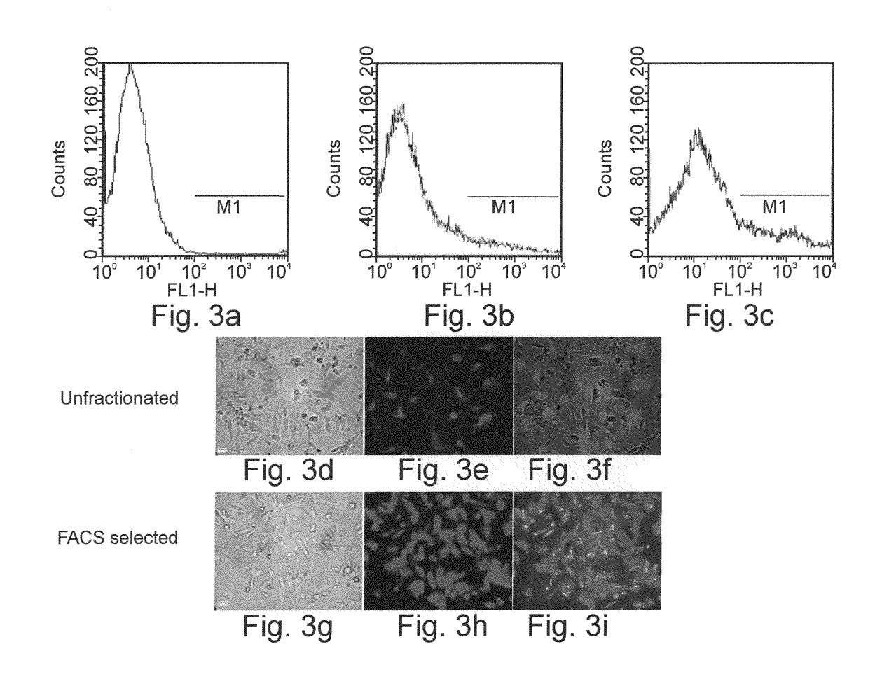

The Examples section below describes an exemplary embodiment of this aspect of the present invention. Following differentiation of a hES cells into a cardiac muscle cell, expression of EGFP is apparent as the tissue specific promoter (e.g. MLC-2v) is activated and enables production of EGFP which can be visualized (e.g. with a fluorescence microscope, FIGS. 2A-C) or separated by a cell sorter (FIGS. 3A-I).

Human ES cells expressing the detectable expression product may be isolated following or concomitant with the detecting such that a purified cell population is generated.

Exemplary methods of isolating cells that express the detectable expression product include, but are not limited to manual dissection (microdissection) of the contracting areas [Kehat, et al., (2002) supra], centrifugation of cells through a Percoll gradient [Xu et al., Circ Res (2002) 91, 501-508], and sorting using a FACS sorter.

As used herein the phrase “purified cell population” refers to a population of human embryonic stem cells wherein at least 80% of the cells therein comprise the same tissue specific phenotype. According to another embodiment, at least 85% of the cells therein comprise the same tissue specific phenotype. According to another embodiment, at least 90% of the cells therein comprise the same tissue specific phenotype. According to another embodiment, at least 95% of the cells therein comprise the same tissue specific phenotype. According to another embodiment, 100% of the cells therein comprise the same tissue specific phenotype.

It will be appreciated that if the tissue specific promoter of the construct of the present invention is a cardiac specific promoter, then the purified cell population will typically comprise a cardiac phenotype.

As used herein the term “cardiac phenotype” refers to either a structural phenotype (e.g. cell morphology) or a functional phenotype (e.g. display of cardiac-specific action potentials, ability to contract, expression of other cardiac markers, or ability to form stable intracardiac cell grafts).

Exemplary cardiac specific markers include, but are not limited to cardiac troponin I (cTnI), sarcomeric α-actinin, MLC-2v, MLC-2a, α-MHC, MEF-2C, and ANF.

As illustrated in the Examples section herein below, purified populations of myocardial cells were generated in which more than 90% of the eGFP-expressing cells were also stained positive for cardiac-specific markers (e.g. sarcomeric α-actinin).

In addition, purity of a cell population may be increased by generation of single cell colonies (generated from a transformed human ES cell). Such single colonies were demonstrated to comprise a higher number cells that both expressed GFP and were stained positive for cardiac-specific markers (e.g. cTnI and MHC), see FIGS. 3B-C.

The cell populations of the present invention may be used to treat diseases. For example, if the cell population of the present invention comprises a population of myocardiocytes, it may be used for treating a myocardial disease.

Thus according to another aspect of the present invention, there is provided a method of treating a myocardial disease. The method comprises administering to the subject a therapeutically effective amount of an isolated cell population which expresses a cardiac phenotype.

As used herein the phrase “myocardial disease” refers to any condition in which there is a deviation from or interruption of the normal structure and/or function of the cardiac tissue or cardiac cells.

Examples of myocardial disease that may be treated according to the teachings of the present invention including ischemic heart disease (IHD) such as angina pectoris, stable angina (typical), variant or Prinzmetal's angina and unstable angina, myocardial infarction (MI), ischemic cardiomyopathy and chronic cardiomyopathy.

As used herein the phrase “a subject in need thereof” refers to a mammal, preferably a human subject who has been diagnosed with or who is susceptible to having a myocardial disease.

The isolated cell population of the present invention are typically from a non-syngeneic source (e.g. allogeneic human ES cells). Since non-syngeneic cells are likely to induce an immune reaction when administered to the body, several approaches have been developed to reduce the likelihood of rejection of non-syngeneic cells. These include either suppressing the recipient immune system or encapsulating the non-autologous cells or tissues in immunoisolating, semipermeable membranes before transplantation.

Encapsulation techniques are generally classified as microencapsulation, involving small spherical vehicles, and macroencapsulation, involving larger flat-sheet and hollow-fiber membranes (Uludag, H. et al. (2000). Technology of mammalian cell encapsulation. Adv Drug Deliv Rev 42, 29-64).

Methods of preparing microcapsules are known in the art and include for example those disclosed in: Lu, M. Z. et al. (2000). Cell encapsulation with alginate and alpha-phenoxycinnamylidene-acetylated poly(allylamine). Biotechnol Bioeng 70, 479-483; Chang, T. M. and Prakash, S. (2001) Procedures for microencapsulation of enzymes, cells and genetically engineered microorganisms. Mol Biotechnol 17, 249-260; and Lu, M. Z., et al. (2000). A novel cell encapsulation method using photosensitive poly(allylamine alpha-cyanocinnamylideneacetate). J Microencapsul 17, 245-521.

For example, microcapsules are prepared using modified collagen in a complex with a ter-polymer shell of 2-hydroxyethyl methylacrylate (HEMA), methacrylic acid (MAA), and methyl methacrylate (MMA), resulting in a capsule thickness of 2-5 μm. Such microcapsules can be further encapsulated with an additional 2-5 μm of ter-polymer shells in order to impart a negatively charged smooth surface and to minimize plasma protein absorption (Chia, S. M. et al. (2002). Multi-layered microcapsules for cell encapsulation. Biomaterials 23, 849-856).

Other microcapsules are based on alginate, a marine polysaccharide (Sambanis, A. (2003). Encapsulated islets in diabetes treatment. Diabetes Thechnol Ther 5, 665-668), or its derivatives. For example, microcapsules can be prepared by the polyelectrolyte complexation between the polyanions sodium alginate and sodium cellulose sulphate and the polycation poly(methylene-co-guanidine) hydrochloride in the presence of calcium chloride.

It will be appreciated that cell encapsulation is improved when smaller capsules are used. Thus, for instance, the quality control, mechanical stability, diffusion properties, and in vitro activities of encapsulated cells improved when the capsule size was reduced from 1 mm to 400 μm (Canaple, L. et al. (2002). Improving cell encapsulation through size control. J Biomater Sci Polym Ed 13, 783-96). Moreover, nanoporous biocapsules with well-controlled pore size as small as 7 nm, tailored surface chemistries, and precise microarchitectures were found to successfully immunoisolate microenvironments for cells (See: Williams, D. (1999). Small is beautiful: microparticle and nanoparticle technology in medical devices. Med Device Technol 10, 6-9; and Desai, T. A. (2002). Microfabrication technology for pancreatic cell encapsulation. Expert Opin Biol Ther 2, 633-646).

The isolated cell population of the present invention can be administered to the subject per se, or as part of a pharmaceutical composition, which also includes a physiologically acceptable carrier. The purpose of a pharmaceutical composition is to facilitate administration of the active ingredient to an organism.

As used herein, a “pharmaceutical composition” refers to a preparation of one or more of the active ingredients described herein with other chemical components such as physiologically suitable carriers and excipients. The purpose of a pharmaceutical composition is to facilitate administration of a compound to an organism.

As used herein, the term “active ingredient” refers to the differentiated embryonic stem cells accountable for the intended biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound.

One may administer a preparation in a local manner, for example, via injection of the preparation directly into a specific region of a patient's body (e.g. cardiac muscle tissue).

A suitable route of administration may, for example, include a direct intraventricular injection.

Pharmaceutical compositions suitable for use in the context of the present invention include compositions wherein the active ingredients are contained in an amount effective to achieve the intended purpose. More specifically, a “therapeutically effective amount” means an amount of active ingredients (e.g., number of cells) effective to prevent, alleviate, or ameliorate symptoms of a disorder (e.g., ischemic heart disease) or prolong the survival of the subject being treated.

Determination of a therapeutically effective amount is well within the capability of those skilled in the art, especially in light of the detailed disclosure provided herein.

For any preparation used in the methods of the invention, the dosage or the therapeutically effective amount can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (See, e.g., Fingl, E. et al. (1975), “The Pharmacological Basis of Therapeutics,” Ch. 1, p. 1.)

Dosage amount and administration intervals may be adjusted individually to provide a sufficient number of cells to induce or suppress the biological effect (i.e., minimally effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks, or until cure is effected or diminution of the disease state is achieved.

The number of cells to be administered will, of course, be dependent on the subject being treated, the severity of the affliction, the manner of administration, the judgment of the prescribing physician, etc.

The purified cell populations of the present invention may also be used to identify novel modulatory agents in a drug screening assay. Thus for example, when the purified cell population comprises cardiac cells, cardiac modulatory agents may be identified. According to this aspect of the present invention, the method comprises contacting the isolated cardiac cell population with an agent, wherein an alteration in a cardiac phenotype of the cell population is indicative of a modulatory effect of the agent.

As used herein the term “cardiac modulatory agent” refers to any agent which is effective in modulating, e.g., enhancing or decreasing, a cardiac phenotype in the cardiac cell. The cardiac agent may be a small molecule, a polypeptide, a peptide a nucleic acid agent.

According to this aspect of the present invention, the contacting is effected under conditions (i.e. for a time long enough or at a suitable temperature) such that the candidate agent is capable of modulating the cardiac phenotype.

Accordingly, any alteration (minor or major) in a cardiac phenotype, including changes in cell morphology, cell function (e.g. ability to contract) and/or changes in expression of cellular markers, may be indicative of a modulatory agent.

It is expected that during the life of a patent maturing from this application many relevant tissue specific promoters will be developed and the scope of the term tissue specific promoters is intended to include all such new technologies a priori.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Generation of Transgenic hESC Lines and Single-Cell Clones

Materials and Experimental Procedures

Generation of Constructs

The constructs generated consisted of the pEGFP-1 vector (Clontech). The EGFP gene was replaced by the HygEGFP gene. The HygR-eGFP fusion protein was under the transcriptional control of either the atrial natriuretic peptide (ANP) or the human myosin light chain-2V (MLC-2v) promoters.

A bluescript plasmid containing a 560 bp fragment of the ANP untranslated region, −470-+90 related to the transcription initiation point (SEQ ID NO: 1) was digested with Sac I and Sal I, and the 560 bp promoter fragment was subcloned to this plasmid, upstream to the HygR-eGFP gene.

A 560 bp fragment of the MLC-2v untranslated region, −513-+47 related to the transcription initiation point (SEQ ID NO: 2), was amplified by polymerase chain reaction using the primers: sense GGAAGATCTGCCACAGTGCCAGCCTTCATGG (SEQ ID NO: 3) and antisense CCCAAGCTTGTGGAAAGGACCCAGCACTGCC (SEQ ID NO: 4), digested with Bgl II and Hind III (restriction sites are in the primers sequence) and subcloned to the above-mentioned HygR-eGFP plasmid.

The vector further contained a second transcriptional unit: SV40 promoter driving the expression of aminoglycoside phosphotransferase—Neo resistance.

For positive control, pEGFP-N1 (Clontech) was used with the Connexin 43 gene. This plasmid expressed the Connexin 43—EGFP fusion protein.

The embryonic stem (ES) cells were transfected using Fugin 6 reagent (Roche) at Fugin to DNA ratio (μl:μg) of 3:2, 3:1, or 6:1.

Electroporation was completed in a Bio-Rad electroporator using the following parameters: 2×10⁶ cells, 40 μg linearized DNA, 320V, 250 μF, 0.4 CM cuvette.

Generation of Lentivirus Constructs

The construct generated consisted of two transcriptional units that were incorporated into a lentiviral vector backbone (pTK113—a self inactivating (SIN) HIV-1 vector).

The first unit included the HygR-eGFP fusion protein under the transcriptional control of either the atrial natriuretic peptide (ANP) or the human myosin light chain-2V (MLC-2v) promoters. A bluescript plasmid containing a 560 bp fragment of the ANP untranslated region, −470-+90 related to the transcription initiation point (SEQ ID NO: 1) was digested with Sac I and Sal I and the 560 bp promoter fragment was subcloned to a plasmid containing the HygR-eGFP gene (based on the pHyg-eGFP from Clontech).

A 560 bp fragment of the MLC-2v untranslated region, −513-+47 related to the transcription initiation point (SEQ ID NO: 2), was amplified by polymerase chain reaction using the primers: sense GGAAGATCTGCCACAGTGCCAGCCTTCATGG (SEQ ID NO: 3) and antisense CCCAAGCTTGTGGAAAGGACCCAGCACTGCC (SEQ ID NO: 4), digested with Bgl II and Hind III and subcloned to the abovementioned HygR-eGFP plasmid. This fragment was chosen due to it homology to a 250 bp fragment in the rat MLC-2 promoter, which was found to be sufficient for cardiac-specific expression [Henderson et al., J Biol Chem (1989) 264, 18142-18148; Zhu et al., Mol Cell Biol (1991) 11, 2273-2281].

The second transcriptional unit contained the PGK promoter driving the expression of aminoglycoside phosphotransferase (PGK-NeoR) which allows selection of the transfected undifferentiated hESC cells. The PGK-NeoR cDNA (SEQ ID NO: 5) was digested from pMSCV Neo (Clontech) using Bgl II and Sal I, and subcloned to pTK113 that was digested with Barn H I and Xho I.

The ANF/MLC-2v-HygR-eGFP and PGK-NeoR fragments were then subcloned to the lentivirus vector pTK113, using Barn HI and XhoI restriction sites.

Establishment of the Transgenic hESC Lines

To generate lentivirus particles, human embryonic kidney (HEK) 293T cells were transfected with 15, 10 or 5 μg of the lentivirus vector, the packaging cassette expression plasmid (ΔNRF) and the VSV-G envelope expression plasmid respectively. HEK 293T cells were transfected using the calcium phosphate transient transfection method. To collect the virus particles, the HEK 293T cell media was harvested 55 hours after transfection and centrifuged at 2000 rpm for 7 minutes. Supernatant was filtered through a 45μ filter and was then concentrated using Vivaspin (membrane cut-off 100,000; Vivascience).

The concentrated virus particles-containing media was supplemented with 6 μg/ml polybrene and was added to the undifferentiated hESC culture medium comprising 20% FBS (HyClone), 80% knockout DMEM (Life Technologies) with 1 mM L-glutamine (Life Technologies), 0.1 mM mercaptoethanol (Life Technologies), and 1% nonessential amino acids (Life Technologies). The undifferentiated hESC cells, clone H9.2 passage 40 [initially clumps of approximately 200 cells obtained at the time of routine passage using mechanical and enzymatic dissociation as was previously described by Amit et al., Dev Biol (2000) 227, 271-278] were incubated for 16 hours with the virus-containing media. The virus particles were collected, added to the hESC culture medium and hESC were infected again as described above. The transduced hESC were then dispersed to small clumps (3-20 cells) using collagenase IV (1 mg/mL, Life Technologies) and re-plated on a fresh mouse feeder layer. These transgenic colonies were isolated and continuously cultured. The transgenic lines that demonstrated robust, stable, long-term and homogenous expression of the transgene were propagated.

Creation of Single-Cell Transgenic hESC Clones

The transgenic hESC colonies established were digested using 0.25 trypsin-EDTA solution (Biological industries; Israel) for 10 minutes. Cells were then counted and diluted to give approximately 1 cell/ml and plated in MEF-covered 24-well plates at a concentration of a single cell per well. The single-cell derived clones were grown as described above and the clones demonstrating robust, stable, long-term and homogenous expression of the transgene were chosen for propagation.

Pluripotent Properties of the Transgenic Lines

To verify that the transgenic hESC lines and clones generated retained the unique properties of the parental hESC lines, the following procedures were performed:

1) Staining for specific hESC markers: undifferentiated stem cells derived from the transgenic lines were stained for specific hESC markers (SSEA-4, Oct-4, Tra-1-60) as described in detail below.

2) Teratoma formation: undifferentiated hESCs were harvested using 1 mg/ml collagenase IV (Life Technologies™) and were injected into the hind-limb of severe combined immunodeficient SCID/beige mice (approximately 5×10⁶ cells per injection). The teratomas were palpable after 6-7 weeks and were harvested for histological examination.

hESC Propagation and In Vitro Cardiomyocyte Differentiation

Undifferentiated transformed hESC were grown on a mitotically inactivated mouse embryonic fibroblast feeder layer (MEF) as previously described [Kehat et al., (2001) supra; Amit et al., supra]. The culture medium consisted of 20% FBS (HyClone), 80% knockout DMEM (Life Technologies) and was supplemented with 1 mM L-glutamine (Life Technologies), 0.1 mM mercaptoethanol (Life Technologies), and 1% nonessential amino acids (Life Technologies).

To induce differentiation, hESC were dispersed to small clumps (3-20 cells) using collagenase IV (1 mg/mL, Life Technologies) and were transferred to plastic Petri dishes at a cell density of about 5×10⁶ cells in a 58 mm dish, where they were cultured in suspension for 7-10 days. During this stage the cells aggregated to form EBs, which were then plated on 0.1% gelatin-coated 24-well plates, at a density of about 7 EBs per well and observed for the appearance of spontaneous contractions.

The contracting areas within the EBs, generated during differentiation, were identified by light microscopy and their presence and location were compared with the spatial distribution of eGFP expression using epifluorescent microscopy by two independent investigators. The eGFP expressing areas were then mechanically dissected for the phenotypic characterization studies described below. In some of these studies, the EBs were dispersed into single cells by enzymatic dissociation as previously described [Satin et al., J Physiol (2004) 559(2):479-496].

Immunostaining

Cells or whole EBs were fixed using 4% paraformaldehyde with sucrose, washed with PBS and permeabilized with 1% Triton-X-100. In the in vivo studies, the hearts were harvested, frozen in liquid nitrogen, and cryo-sectioned.

Cells or tissue sections were then blocked with 5% normal goat serum or horse serum and incubated overnight at 4° C. with primary antibodies for cardiac troponin I (cTnI, Chemicon), connexin-43 (Cx43, Chemicon), human mitochondria (Chemicon), sarcomeric a-actinin (Sigma), MLC-2v (Santa-Cruz), Oct-4 (Santa-Cruz), SSEA-4 (Chemicon) and Tra-1-60 (Chemicon). Secondary antibodies, Cy3- and Cy2-conjugated donkey and goat anti-mouse IgG antibodies and Cy3-, Cy2- and Cy5-conjugated anti-rabbit IgG antibodies (Jackson), were diluted 1:200 and were incubated for one hour at room temperature. Nuclei were counterstained by ToPro3 (Molecular Probes) or DAPI (Sigma).

To overcome possible autofluorescence artifact at the injection site, additional immunostainings was carried out using polyclonal antibodies for eGFP (1:100, MBL) in a similar manner as described above. Accordingly, the above mentioned secondary antibodies were utilized (with different excitation/emission spectra). Furthermore, negative control staining was performed in which the secondary antibodies were added without the primary antibody, in an attempt to assess the degree of autofluorescence.

Confocal microscopy was performed using a Nikon Eclipse E600 microscope and Bio-Rad Radiance 2000 scanning system.

Results

The generated constructs, each containing two transcriptional units, were incorporated into a self-inactivating lentiviral vector backbone (pTK113). The first unit included the phosphoglycerate kinase promoter driving the expression of aminoglycoside phosphotransferase (PGK promoter-NeoR) cassette and was used to achieve stable transfection and selection of the undifferentiated hESC carrying the vector. The second unit contained a cardiac-restrictive promoter (the human MLC-2v or ANP) driving the expression of the HygR-eGFP fusion protein cassette that allowed identification and selection of the generated cardiomyocytes.

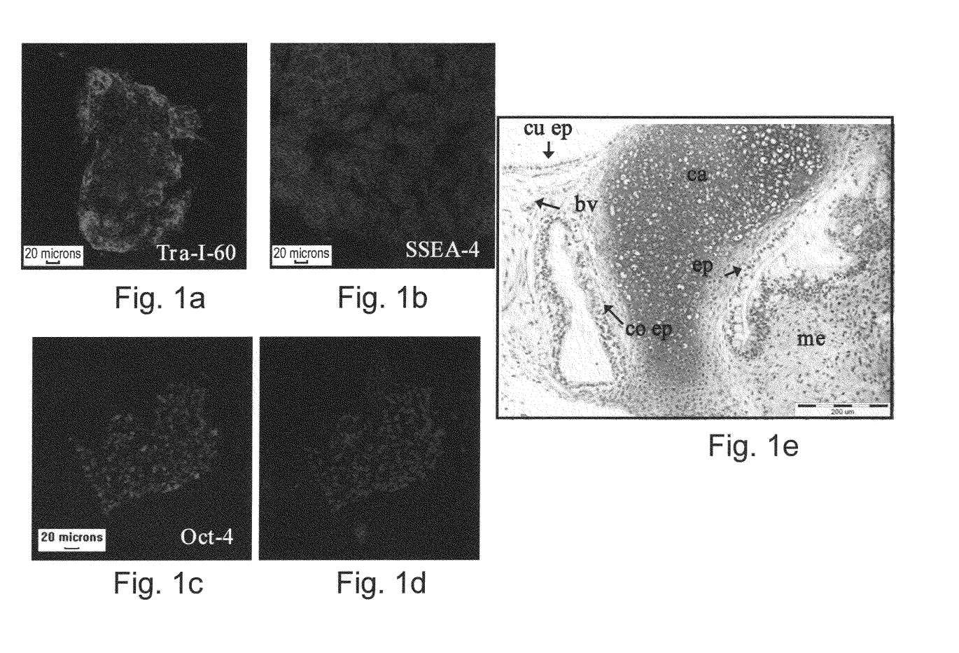

The inventors tried various methods of transfection of the pEGFP-1 vectors including Fugin 6 reagent, electroporation, FUGIN HD and jet Pei, however none of these methods worked (data not shown). In addition, the present inventors used other viral vectors (e.g. pHIV puro-X-M) in order to infect the human embryonic stem cells with the constructs of the present invention. Only the aforementioned lentiviral vectors and calcium phosphate transient transfection, were successful in infecting the human embryonic stem cells such that a long-term stable expression of the transgene was effected. Accordingly, 7 stable transgenic hESC lines were generated (5 using the MLC-2v promoter and 2 using the ANP promoter). PCR analysis of selected colonies confirmed the presence of both transcriptional units in the transfected lines (data not shown). The transgenic hESC lines were analyzed and compared to the parental lines from which they were derived. No significant differences were found in their immunostaining results for the presence of typical undifferentiated hESC markers (Oct-4, SSEA-4 and Tra-1-60, FIGS. 1A-D) and their ability to form teratomas when injected into SCID mice (FIG. 1E).

Next, the established transgenic hESC lines were utilized to identify and select for the differentiating cardiomyocytes. Following selection and expansion of the transfected undifferentiated colonies, the hESC were allowed to differentiate using the EB differentiating system as previously described [Kehat et al. (2001), supra]. Thus, as described in the materials and methods section above, following 7-10 days of cultivation in suspension, the EBs were plated on gelatin-coated culture plates and observed using light and epifluorescent microscopy for the appearance of spontaneous contraction and eGFP expression respectively.

Stable transfection of the hESC lines did not affect their cardiomyocyte differentiating capabilities with approximately 10-15% of the EBs showing spontaneous contracting areas (in a similar manner to the wild-type lines). An excellent spatial correlation was noted between the location of the beating areas in the contracting EBs and the presence of eGFP expression (FIGS. 2A-C). Hence, 91% of the 899 EBs that were scanned and demonstrated to contain contracting zones in the MLC-2V-transgenic lines also displayed positive eGFP fluorescence in the same regions (ranging from 67% to 98% in the 5 different MLC-2v lines). More importantly, prospective analysis of 129 EBs showing condensed eGFP fluorescence demonstrated that 98% also displayed spontaneous beating in the same areas. Importantly, eGFP expression could also be noted and maintained in differentiating EBs (undergoing long-term culturing) that were derived from transgenic hESC lines that have undergone multiple passages (31 passages, the longest period analyzed). This indicated the lack of promoter shut down (which may be a significant limiting factor in the genetic modification of hESC) in the transgenic lines that were chosen for propagation. Likewise, in the two ANP-transgenic lines the percentage of the beating EBs containing eGFP expressing areas was 41% and 93%. Interestingly, expression of eGFP was already apparent in the MLC-2v- and ANP-hESC lines at 1-2 days prior to the initiation of spontaneous contractions and in MLC-2v hESC lines eGFP expression was continuous for up to 50 days post-plating (the longest period analyzed).

The observed variability between the different hESC transgenic lines generated may have resulted from the fact that the established lines were not truly single-cell clones but rather were derived from individually transfected colonies and, hence, not all cells in the differentiating EBs may have shown the same level of transgene expression. To test this hypothesis, single-cell clones were further generated from one of the transgenic MLC-2V lines. A total of 14 single-cell clones were established, of which 3 were continuously propagated (those that displayed continuous, homogeneous, and robust eGFP fluorescence during EB differentiation).

The single-cell clones were characterized by the same unique undifferentiated properties, pluripotency and capacity to differentiate into cardiomyocytes as the parental lines. Yet, they were also characterized by a more homogeneous expression of the transgene during in vitro EB differentiation. This was manifested by an increase in the percentage of beating EBs showing eGFP fluorescence (100% in the single-cell clones vs. 67% to 98% in the regular transgenic lines). Similarly, the pattern and level (intensity) of eGFP fluorescence within a single beating area was more homogeneous in the EBs generated from the single-cell clones when compared to the parental transgenic hESC line (FIGS. 2A-J).

### Example 2

### Characterization of the eGFP Expressing Cells

Materials and Experimental Procedures

Immunostaining

As described in Example 1, above.

FACS Sorting

EBs were digested using 0.25 trypsin-EDTA solution (Biological industries, Israel) for 10 minutes. Due to the need for a relatively large number of eGFP cells for the FACS sorting studies, EBs were taken from wells showing an increased rate of contraction. In other studies, aiming to examine the entire population of eGFP-expressing cells, the entire population of differentiating EBs was used. Cells were then re-suspended in the culture medium at a concentration of 10⁶ cells/ml.

Flow cytometric analysis was performed using a FACS sorter (Becton Dickinson Immunocytometry Systems, USA). A 530/30 nm bandpass filter was used to measure eGFP fluorescence intensity excited with the 488 nm line of an argon ion laser. Detector settings were calibrated with untransfected hESC derived EBs that were digested by the same method. The FACS sorted cells were plated on gelatin coated 24-wells culture plates at a density of 10⁵ cells/well.

RT-PCR Analysis

Total RNA was isolated from undifferentiated hESC, unfractionated dispersed cells derived from the differentiating EBs, FACS-sorted eGFP-expressing cells and the non-sorted cells using the high-pure RNA isolation kit (Roche). cDNA was synthesized using access RT-PCR introductory system (Promega) and subjected to PCR with primers for cardiac specific genes (GATA 4, ANF and MEF2C), pluripotent markers (Oct4), endodermal (a-fetoprotein), ectodermal (beta-III-tubulin), and β actin (see Table 1, below)

TABLE 1
Primers and reaction conditions used in the RT-PCR studies

| Gene | Primer | Tm |
|---|---|---|
| GAPDH | sense- AGCCACATCGCTCAGACACC | 60° C. |
| | (SEQ ID NO: 6) | |
| | anti-sense- GTACTCAGCGGCCAGCATCG | |
| | (SEQ ID NO: 7) | |
| Oct-4 | sense- GAGAACAATGAGAACCTTCAGGAGA | 60° C. |
| | (SEQ ID NO: 8) | |
| | antisense- TTCTGGCGCCGGTTACAGAACCA | |
| | (SEQ ID NO: 9) | |
| α-fetoprotein | sense- AGAACCTGTCACAAGCTGTGAA | 60° C. |
| | (SEQ ID NO: 10) | |
| | antisense- GACAGCAAGCTGAGGATGTCT | |
| | (SEQ ID NO: 11) | |
| MLC-2a | sense- AAGGTGAGTGTCCCAGAGG | 56° C. |
| | (SEQ ID NO: 12) | |
| | antisense- ACAGAGTTTATTGAGGTGCCC | |
| | (SEQ ID NO: 13) | |
| MLC-2v | sense- TATTGGAACATGGCCTCTGGAT | 58° C. |
| | (SEQ ID NO: 14) | |
| | antisense- GGTGCTGAAGGCTGATTACGTT | |
| | (SEQ ID NO: 15) | |
| α-MHC | sense- GTCATTGCTGAAACCGAGAATG | 58° C. |
| | (SEQ ID NO: 16) | |
| | antisense- GCAAAGTACTGGATGACACGCT | |
| | (SEQ ID NO: 17) | |
| β III tubulin | sense- CAGGCCTGACAATTTCATCTTTG | 62° C. |
| | (SEQ ID NO: 18) | |
| | antisense- ACCATGTTGACGGCCAGCTTG | |
| | (SEQ ID NO: 19) | |
| β actin | sense- CTGGAACGGTGAAGGTGACA | 60° C. |
| | (SEQ ID NO: 20) | |
| | antisense- CAATGCTATCACCTCCCCTGT | |
| | (SEQ ID NO: 21) | |
| GATA 4 | sense- GACGGGTCACTATCTGTGCAAC | 60° C. |
| | (SEQ ID NO: 22) | |
| | antisense- AGACATCGCACTGACTGAGAAC | |
| | (SEQ ID NO: 23) | |
| ANF | sense- GAACCAGAGGGGAGAGACAGAG | 60° C. |
| | (SEQ ID NO: 24) | |
| | antisense- CCCTCAGCTTGCTTTTTAGGAG | |
| | (SEQ ID NO: 25) | |
| MEF2C | sense- GAACAATCCCGGTGTGTCAGGA | 60° C. |
| | (SEQ ID NO: 26) | |
| | antisense- CACCCAGTGGCAGCCTTTTACA | |
| | (SEQ ID NO: 27) | |

Patch-Clamp Studies

For single cell action-potential analysis, the whole-cell configuration of the patch-clamp technique was used as previously described (Satin et al., supra). After dissociation with collagenase B (1 mg/mL, Roche), cells were re-plated for 1-3 days on gelatin-coated glass coverslips. The patch pipette solution consisted of: 120 mM KCl, 1 mM MgCl2, 3 mM Mg-ATP, 10 mM Hepes, 10 mM EGTA, pH-7.3. The bath recording solution consisted of: 140 mM NaCl, 5.4 mM KCl, 1.8 mM CaCl₂, 1 mM MgCl₂, 10 mM Hepes, 10 mM glucose, pH 7.4. Upon seal formation and following patch-break analog capacitance compensation was used. Axopatch 200B, Digidata1322, and pClamp8 (Axon, Burlingame, Calif.) were used for data amplification, acquisition, and analysis. A cardiac phenotype was assigned to the examined cells if it displayed cardiac action potential or ionic currents in the current-clamp or voltage-clamp modes respectively. A total of 33 eGFP-cells were studied.

Multi-Electrode Array Recordings

The electrophysiological properties of the eGFP-expressing cell-clusters were examined using a microelectrode array (MEA) data acquisition system (Multichannel Systems, Reutlingen, Germany) as previously described (Kehat et al., (2002), supra; Feld et al., Circulation (2002) 105, 522-529). The MEA plates consisted of a matrix of 60 electrodes with an interelectrode distance of 100 or 200 μm allowing simultaneous recording of the extracellular potentials at a sampling rate of 10 KHz. All recordings were performed at 37° C. and a pH of 7.4. Local activation time (LAT) at each electrode was determined by the timing of the maximal negative intrinsic deflection (dV/dtmin). This information was then used for the generation of color-coded activation maps by interpolating the LAT values between the electrodes using MATLAB standard two-dimensional plotting function.

Results

Immunostaining studies of the EBs (FIGS. 2A-J) demonstrated that the eGFP-expressing cells were stained positive for cardiac-specific markers. For example, as illustrated in FIGS. 2A-C at both low (FIG. 2B) and high (FIG. 2C) magnifications, the cells expressing eGFP within the EBs were also stained positive with anti-cTnI antibodies. Moreover, these cells demonstrated an early-striated pattern, typical of early-stage cardiomyocytes. The cardiac specificity of the MLC-2v promoter-driven eGFP expression could be more clearly demonstrated in immunocytostaining studies of dispersed cells, isolated from the beating areas (FIGS. 2D-E). Quantitative analysis of these co-localization immunocytostaining experiments (see Table 2, below) demonstrated that 91% (429/474, n=5) of the eGFP-expressing cells were also stained positive for cardiac-specific markers (using anti-sarcomeric α-actinin antibodies). In contrast, about 14% of the α-actinin positive cells were eGFP negative.

TABLE 2
Co-localization studies of eGFP expression and immunostaining
for cardiac-specific marker in dispersed cells isolated
from the differentiating EBs

| | eGFP | eGFP |
|---|---|---|
| | positive cells | negative cells |
| Positive staining for α-actinin | 429 | 70 |
| Negative staining for α-actinin | 45 | 153 |

The EBs derived from the single-cell clones were characterized by a more intense and homogeneous eGFP expression compared to the parental transgenic hESC lines (FIGS. 2F-J). The eGFP-expressing cells derived during the differentiation of these single-cell clones were stained positive for cardiac-specific markers (cTnI and MHC) either as dispersed cells (FIGS. 2F-H) or as whole EBs (FIGS. 2I-J).

Subsequently, strategies were developed for the selection of the eGFP-expressing cells. To this end, FACS sorting of dispersed cells (obtained using enzymatic dissociation of the differentiating EBs) was used to select for the eGFP-expressing cells (FIGS. 3A-I). These studies also demonstrated a significant improvement in the single-cell clones over the parental polyclonal lines (FIG. 3C vs. FIG. 3B). Thus, pooled FACS analysis characterizing the differentiating EBs demonstrated that the number of eGFP-expressing cells (normalized per a single beating EB) was higher in the single-cell clone versus the parental transgenic line (339 and 169 eGFP-expressing cells, respectively).

An analysis of the viability of the cells following the FACS sorting procedure was accomplished using trypan blue staining. The results showed that 95% of the cells were viable prior to FACS sorting (following the enzymatic dispersion) while 85% of the sorted eGFP-cells remained viable following this FACS procedure (data not shown).

The selected eGFP-expressing cardiomyocytes were then maintained and remained viable for several weeks in culture (FIGS. 3G-I). These eGFP-expressing cells were stained positive for different cardiac-specific markers including anti-MLC-2v antibodies (FIGS. 4A-C). Moreover, the fraction of cells that continued to express eGFP following the FACS selection procedure and the percentage of these cells that express cardiac-specific markers was quantified. The results indicated that 96.8% (244 out of 252, n=4) of the sorted cells continued to express eGFP and that 93.4% (228/244, n=4) of these cells were also stained positive for cardiac-specific markers. In contrast, plating of the unfractionated dispersed cells, derived from similar stage contracting EBs, without FACS selection, resulted in the majority of these cultured cells not having a myocyte phenotype (FIGS. 3D-F). Moreover, the eGFP-based FACS selection strategy was found to be significantly better (p<0.01) than that of microdissection of the contracting areas from wild-type EBs (n=6), in which only 58.8% of the cells were found to be positively stained for cardiac-specific markers.

Similar to the immunostaining studies, RT-PCR studies of the selected eGFP-cells demonstrated the expression of cardiac-specific genes. FIG. 4D depicts the results of these RT-PCR studies in four populations of cells: undifferentiated hESC, unfractionated cells derived from the differentiating EBs (prior to FACS), the sorted eGFP-expressing cells and the GFP-negative cells. Note, the expression of the pluripotent marker, Oct4, in the undifferentiated hESC and its significant down-regulation in all differentiated progeny. Also note that the highest expression of the cardiac-specific genes (MLC-2v, MLC-2a and α-MHC) was found in the eGFP-sorted population with a lower degree of expression in the unfractionated cells and even a lower degree in the non-sorted population (FIG. 4D). Similarly, the expression of non-myocyte markers, such as α-fetoprotein (an endodermal marker) and beta-III-tubulin (an ectodermal marker), could be identified in the unfractionated population with a significant diminution in the eGFP-selected cells.

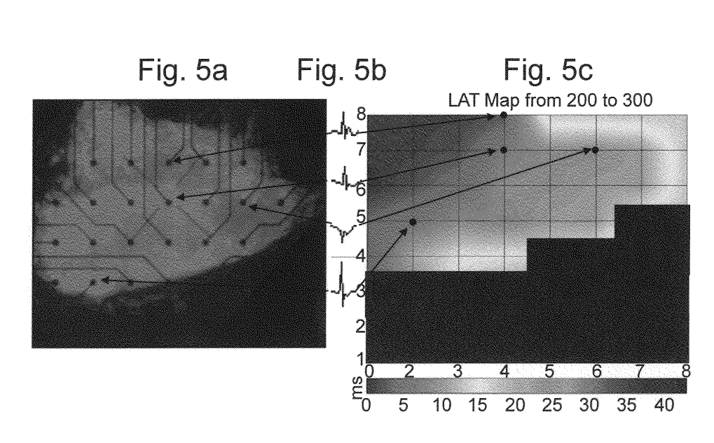

Next, it was determined whether the eGFP-expressing cells also demonstrated functional properties typical of cardiomyocytes. The eGFP-expressing areas within the EBs were mechanically dissected and plated on top of a microelectrode array (MEA) mapping technique (FIG. 5A). The MEA, comprised of 60 electrodes (spaced 100 μm apart), allowed assessment of the electrical activity with extremely high spatial and temporal resolutions. An excellent spatial correlation was noted between the location of the eGFP-expressing area in the EB and the recording of electrical activity. Hence, local extracellular potentials could be recorded only in electrodes directly underlying the eGFP-expressing cells (FIGS. 5A-C). Determination of the LAT at each electrode allowed the construction of detailed activation maps depicting the spread of electrical activation within the eGFP-expressing region (FIG. 5C). These studies also showed that both the areas initiating the electrical activity (pacemaker areas) as well as the areas in which the action potential was propagated consisted of eGFP-expressing cells.

Whole cell patch-clamp studies of the eGFP-expressing cells, either in small clumps or as isolated cells, demonstrated the presence of cardiac-specific action-potentials (FIGS. 6A-E). Interestingly, the presence of a cardiac-specific electrophysiological signature was also observed in eGFP-expressing cells that were not beating spontaneously. Out of the 33 eGFP-expressing cells studied, 32 were determined to be cardiomyocytes, based on either the presence of cardiac-specific action-potentials or ionic transients using the current- or voltage-clamp modes respectively.

The action-potential properties of the eGFP-expressing cells were compared with the cardiomyocytes isolated from similar stage EBs derived from wild-type hESC lines (FIGS. 6D-E). Hence, the transfection of the eGFP expressing cells did not significantly effect the action-potential morphologies recorded from these cells and wild-type cells at similar developmental stages. In all cases an “embryonic”-like phenotype was identified. The action-potential measurements were also comparable between the cells with the maximal diastolic potentials (MDP) recorded being: −54.2±3.2 mV and −55.3±5.1 mV in the MLC-2V and wild-type derived hESC derived cardiomyocytes, respectively, and APD90 averaging 253±33 ms and 288±65 ms, respectively.

### Example 3

### In Vivo Grafting

Materials and Experimental Procedures

Myocardial Engraftment of the eGFP-Expressing Cells

All animal experiments were approved by the Animal Board and Safety Committee of the Technion's Faculty of Medicine. For the transplantation studies, the eGFP-expressing areas within the differentiating EBs (20-40 days of differentiation) were carefully micro-dissected with a curved 23G needle and were then dissociated into small cell clusters (20-100 cells) by incubation with 1 mg/ml of collagenase B (Roche) for 45 minutes. This protocol resulted in the best survival rate of the grafted cells.

Male Sprague-Dawley rats weighing 200-250 gr were anesthetized using a ketamine/xylasin preparation and mechanically ventilated with a Harvard small-animal mechanical respirator. Through a left thoracotomy, the eGFP-expressing cells clusters were grafted to a left ventricular site using a 28 g needle (a suture was used to mark the exact locations where injections were made). The cells were suspended prior to injection in 300 μL serum-free media. Following the procedure, the animals were treated by daily injections of cyclosporine-A (10 mg/kg) and methylprednisolone (2 mg/kg) to prevent immune rejection. Three days or four weeks following cell grafting, the hearts were harvested for pathological examination.

Results

Proof-of-concept studies were performed to test the ability of the eGFP-expressing cells, derived from the MLC-2V transgenic line, to form stable intracardiac cell grafts. Three days (n=4) and four weeks (n=3) following cell grafting, the animals were sacrificed and their hearts were harvested for pathological examination. As can be seen in FIGS. 7A-J, the grafted cells survived and could be identified in all animals studied as relatively small, eGFP-expressing cells that were interspersed isotropically within host rat myocardium. The cardiomyocyte and human phenotype of the grafted eGFP-expressing cells was verified by co-staining for cardiac-specific (FIGS. 7B-C) and human-specific (FIGS. 7E-F) markers, respectively. Quantitative assessment of the histological specimens (n=4) demonstrated that 95% of the eGFP-expressing cells (273/287 cells) were also stained positive for cardiac-specific markers. Immunostaining studies for both undifferentiated markers (Oct-4) and endodermal markers (a-fetoprotein) failed to show any positive staining within the cell graft.

The transplanted cells could still be identified within the host myocardium as long as 4 weeks following cell grafting (the longest period studied, FIGS. 7G-J). Interestingly, there seemed to be some form of structural maturation during this period with the grafted cells. The cells increased in size and showed a more elongated morphology (FIGS. 7G-I). Importantly, the grafted cells formed gap junctions with host myocardial cells (positive punctuated immunostaining for Cx43 indicated by the arrows in FIG. 7J).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A nucleic acid construct comprising a polynucleotide comprising a nucleic acid sequence encoding a detectable expression product, said nucleic acid sequence being operably linked to a human tissue specific promoter.

2. The nucleic acid construct of claim 1, further comprising an additional polynucleotide comprising a nucleic acid sequence encoding an antibiotic resistance moiety, said nucleic acid sequence being operably linked to a constitutive promoter.

3. The nucleic acid construct of claim 1, wherein said human tissue specific promoter comprises a cardiac specific promoter.

4. The nucleic acid construct of claim 3, wherein said cardiac specific promoter comprises a myosin light-chain-2 (MLC-2v) promoter.

5. The nucleic acid construct of claim 3, wherein said cardiac specific promoter comprises an atrial natriuretic peptide (ANP) promoter.

6. The nucleic acid construct of claim 1, wherein the construct comprises a lentivirus backbone.

7. The nucleic acid construct of claim 6, wherein said lentivirus backbone comprises a PTK 113 backbone.

8. An isolated human embryonic stem cell comprising the nucleic acid construct of claim 1.

9. A purified cell population comprising human embryonic stem cells expressing the nucleic acid construct of claim 1.

10. The purified cell population of claim 9, wherein said human embryonic stem cells comprises a cardiac phenotype.

11. The purified cell population of claim 10, wherein said cardiac phenotype comprises a functional phenotype.

12. The purified cell population of claim 11, wherein said functional phenotype comprises an expression of a cardiac marker.

13. The purified cell population of claim 12, wherein said cardiac marker is selected from the group consisting of cardiac troponin I (cTnI), sarcomeric α-actinin, MLC-2v, MLC-2a, α-MHC, MEF-2C and ANF.

14. A method of lineage tracing of human stem cells comprising:
(a) introducing the nucleic acid construct of claim 1, into human embryonic stem (ES) cells;
(b) culturing said human ES cells under conditions which allow differentiation into a tissue lineage, and
(c) detecting expression of said detectable expression product, thereby lineage tracing the human stem cells.

15. The method of claim 14, further comprising isolating cells exhibiting said expression of said detectable expression product.

16. An isolated population of cells generated according to the method of claim 15.

17. The isolated population of cells of claim 16, wherein said human tissue specific promoter comprises a cardiac specific promoter.

18. A method of treating a myocardial disease in a subject in need thereof, the method comprising administering to the subject a therapeutically effective amount of the isolated cell population of claim 17, thereby treating the myocardial disease in the subject.

19. (canceled)

20. A method of identifying a cardiac modulatory agent, the method comprising contacting the isolated cell population of claim 17 with an agent, wherein an alteration in a cardiac phenotype of the cell population is indicative of a modulatory effect of said agent, thereby identifying the cardiac modulatory agent.

21. A method of lineage tracing of human stem cells comprising:
(a) introducing the nucleic acid construct of claim 4, into human embryonic stem (ES) cells;
(b) culturing said human ES cells under conditions which allow differentiation into a tissue lineage, and
(c) detecting expression of said detectable expression product, thereby lineage tracing the human stem cells.

22. A method of lineage tracing of human stem cells comprising:
(a) introducing the nucleic acid construct of claim 5, into human embryonic stem (ES) cells;
(b) culturing said human ES cells under conditions which allow differentiation into a tissue lineage, and
(c) detecting expression of said detectable expression product, thereby lineage tracing the human stem cells.


SEQUENCE LISTING (27 sequences)

SEQ ID NO: 1 - DNA, 560 bp, Artificial sequence
  A polynucleotide fragment derived from the human ANP promotor region
atccatttgtctcgggctgctggctgcctgccatttcctcctctccacccttatttggaggccctgacagctgagccacaaacaaaccaggggagctgggcaccagcaagcgtcaccctctgtttccccgcacggtaccagcgtcgaggagaaagaatcctgaggcacggcggtgagataaccaaggactcttttttactcttctcacacctttgaagtgggagcctcttgagtcaaatcagtaagaatgcggctcttgcagctgagggtctggggggctgttggggctgcccaaggcagagaggggctgtgacaagccctgcggatgataactttaaaagggcatctcctgctggcttctcacttggcagctttatcactgcaagtgacagaatggggagggttctgtctctcctgcgtgcttggagagctggggggctataaaaagaggcggcactgggcagctgggagacagggacagacgtaggccaagagaggggaaccagagaggaaccagaggggagagacagagcagcaagcagtggattgctccttgacga

SEQ ID NO: 2 - DNA, 561 bp, Artificial sequence
  A polynucleotide fragment derived from human the MLC-2v promotor region
gccacagtgccagccttcatggttattttaaagatggtggtcggggaggacttcagctcaggagatacatatgagcaaagatgcagtgaaggaggtgaaggaaggagccgtgcgatgactgacagaaagacattccaggtagagggacacaggtgcaaagaccctgaggccagatccaggctgataaaacagagccattttagcagtctcctctccctgccattttttttctcaaaattgacaaggcacaagtgtccccggcccaacacccagagagcagcagcatctctccccgtgaccatgacccagctactgcctctttaaccttgaatgcctttttgggggctcacgtgtcacccagtggcgagtgagccacccttacttcagaagaacggcatggggtgggggggccttaggtggtgcccgcctcacctatgactgccaaaagcggtcatggggttatttttaaacatggggaggaagtatttattgttcctgggctgcagagagctgggcggagtgtggaattcttgtcgggaggcagtgctgggtcctttccac

SEQ ID NO: 3 - DNA, 31 bp, Artificial sequence
  Single strand DNA oligonucleotide
ggaagatctgccacagtgccagccttcatgg

SEQ ID NO: 4 - DNA, 31 bp, Artificial sequence
  Single strand DNA oligonucleotide
cccaagcttgtggaaaggacccagcactgcc

SEQ ID NO: 5 - DNA, 1325 bp, Artificial sequence
  PGK-NeoR cDNA sequence
attctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 6 - DNA, 20 bp, Artificial sequence
  Single strand DNA oligonucleotide
agccacatcgctcagacacc

SEQ ID NO: 7 - DNA, 20 bp, Artificial sequence
  Single strand DNA oligonucleotide
gtactcagcggccagcatcg

SEQ ID NO: 8 - DNA, 25 bp, Artificial sequence
  Single strand DNA oligonucleotide
gagaacaatgagaaccttcaggaga

SEQ ID NO: 9 - DNA, 23 bp, Artificial sequence
  Single strand DNA oligonucleotide
ttctggcgccggttacagaacca

SEQ ID NO: 10 - DNA, 22 bp, Artificial sequence
  Single strand DNA oligonucleotide
agaacctgtcacaagctgtgaa

SEQ ID NO: 11 - DNA, 21 bp, Artificial sequence
  Single strand DNA oligonucleotide
gacagcaagctgaggatgtct

SEQ ID NO: 12 - DNA, 19 bp, Artificial sequence
  Single strand DNA oligonucleotide
aaggtgagtgtcccagagg

SEQ ID NO: 13 - DNA, 21 bp, Artificial sequence
  Single strand DNA oligonucleotide
acagagtttattgaggtgccc

SEQ ID NO: 14 - DNA, 22 bp, Artificial sequence
  Single strand DNA oligonucleotide
tattggaacatggcctctggat

SEQ ID NO: 15 - DNA, 22 bp, Artificial sequence
  Single strand DNA oligonucleotide
ggtgctgaaggctgattacgtt

SEQ ID NO: 16 - DNA, 22 bp, Artificial sequence
  Single strand DNA oligonucleotide
gtcattgctgaaaccgagaatg

SEQ ID NO: 17 - DNA, 22 bp, Artificial sequence
  Single strand DNA oligonucleotide
gcaaagtactggatgacacgct

SEQ ID NO: 18 - DNA, 23 bp, Artificial sequence
  Single strand DNA oligonucleotide
caggcctgacaatttcatctttg

SEQ ID NO: 19 - DNA, 21 bp, Artificial sequence
  Single strand DNA oligonucleotide
accatgttgacggccagcttg

SEQ ID NO: 20 - DNA, 20 bp, Artificial sequence
  Single strand DNA oligonucleotide
ctggaacggtgaaggtgaca

SEQ ID NO: 21 - DNA, 21 bp, Artificial sequence
  Single strand DNA oligonucleotide
caatgctatcacctcccctgt

SEQ ID NO: 22 - DNA, 22 bp, Artificial sequence
  Single strand DNA oligonucleotide
gacgggtcactatctgtgcaac

SEQ ID NO: 23 - DNA, 22 bp, Artificial sequence
  Single strand DNA oligonucleotide
agacatcgcactgactgagaac

SEQ ID NO: 24 - DNA, 22 bp, Artificial sequence
  Single strand DNA oligonucleotide
gaaccagaggggagagacagag

SEQ ID NO: 25 - DNA, 22 bp, Artificial sequence
  Single strand DNA oligonucleotide
ccctcagcttgctttttaggag

SEQ ID NO: 26 - DNA, 22 bp, Artificial sequence
  Single strand DNA oligonucleotide
gaacaatcccggtgtgtcagga

SEQ ID NO: 27 - DNA, 22 bp, Artificial sequence
  Single strand DNA oligonucleotide
cacccagtggcagccttttaca